(19) United States
(12) Patent Application Publication
(10) Pub. No.: US 2010/0292794 A1
(43) Pub. Date: Nov. 18, 2010

(54) Device for implanting in a human or animal vertebral column

(76) Inventor: Peter Metz-Stavenhagen, Bad Wildungen (DE)

Correspondence Address: Thomas R. Vigil Law Offices, 319 Bluff Court, Barrington, IL, 60010, US

(21) Appl. No.: 10/557,388

(22) PCT Filed: May 19, 2004
(86) PCT No.: PCT/DE04/01060

§ 371 Date: Nov. 15, 2006

(30) Foreign Application Priority Data
May 19, 2003 (DE) 203 07 876.4

(51) Int. Cl. A61F 2/44

(52) U.S. Cl. 623/17.11

(57) ABSTRACT

The invention relates to a device for implanting in a human or animal vertebral column, said device comprising a base carrier (10) that can be fixed to a first vertebral body, an upper carrier (14) that can be fixed to a second vertebral body, and a spacer element fixed between the base carrier and the upper carrier. The base carrier (10) and/or the upper carrier (14) are provided with a cavity (18, 24) comprising undercuts, for receiving the spacer element in a positively locked manner. The aim of the invention is to economically create one such device that ensures complete freedom of movement of the vertebral column. To this end, the spacer element is embodied as metallic bellows (12).

DE 203 07 876.4 dated May 19, 2003

### BACKGROUND OF THE INVENTION

1. Field of the Invention

The present invention relates to a device for implantation into a human or animal spine according to the preamble of claim 1. The human spine is formed from various vertebral bodies that are separated from each other by an intervertebral disc (simply called disc in common parlance) acting as a cushion therebetween. These intervertebral discs are made up of a fibrous outer casing that contains a gelatinous liquid. The intervertebral discs are subjected to high forces that may vary from about 780 N to no less than about 2200 N. In particular with age, these high stress forces often cause wear on the intervertebral discs. A once destroyed intervertebral disc cannot heal so that damaged intervertebral discs are usually removed and the adjacent vertebral bodies fused together. The disadvantage thereof is that in this region the spine will be stiff.

2. Description of the Prior Art

DE 38 73 566 T2 suggests to separate adjacent vertebral bodies by an implant comprising a spring for keeping the spine mobile in this region. This implant consists of a base carrier and a top carrier that are fixated each on corresponding sides of the vertebral bodies and of an elastic element consisting of two helical springs that is retained on the base carrier and the top carrier. Concurrently, the base carrier and the top carrier are connected together through a common axis in such a manner that both are pivotal about said axis. With this implant, adjacent vertebral bodies remain mobile in the pivoting direction so that a patient provided with such an implant is again capable of stooping. Unfortunately, the rigid pivot axis makes a movement to the right or to the left impossible so that the freedom of movement of a patient provided with such an implant is restricted.

The implant according to DE 38 73 566 T2 consists of a plurality of individual parts which makes it very costly to produce. Moreover, the many individual parts involve the risk of tissue ingrowth therein, which results in pain or other disabilities.

From DE 43 15 757 C1 an implant is known that comprises a resilient intermediate element. This intermediate element is composed of a number of substantially cylindrically configured discs that are elastic in the axial direction with the outer wall of the disc being configured to be either concave or convex in order to thus resiliently absorb the forces generated in the axial direction. However, the connections between adjacent discs are hereby subjected to very high loads that may cause damages. Moreover, the implants are made from one piece so that a complete implant must be kept in store for each application case, which results in considerable costs.

An implant is known from U.S. Pat. No. 5,370,697 that is built in a modular fashion. This implant is composed of an upper and a lower element with a non-elastic tube being insertable therein between to act as a separator in order to span the missing disc or the missing vertebral bone. The elasticity of the spine gets lost in the process.

### BRIEF SUMMARY OF THE INVENTION

In view thereof, it is the object of the present invention to provide a device for implantation in a human or animal spine that ensures at low manufacturing cost complete freedom of movement of the spine.

The technical solution to this object proposed by the invention is a device having the features of claim 1. Advantageous developed implementations of this device are recited in the subordinate claims.

A device for implantation in a human or animal spine configured according to this technical teaching has the advantage that the elastic element, which is configured in the form of an undulated bellows, is pivotal in any direction while exhibiting sufficient stiffness to obviate the need for further connection between the base carrier and the top carrier. The undulated bellows further has the advantage that bending may occur in the desired direction without high forces acting thereby onto the base carrier and/or the top carrier. AS a result, the load on the screws fixing the base carrier and/or the top carrier is kept low.

In a preferred embodiment, undercuts, more specifically in the form of a dovetail joint, are provided in the base carrier and/or in the top carrier on the side turned toward the undulated bellows for interlockingly receiving the parts of the undulated bellows. It is thereby readily possible to for example first introduce the undulated bellows into the base carrier in such a manner that the last undulation is caused to fit the receiving portion. The top carrier may then also be pulled in such a manner over the thus fixated undulated bellows that the crest of the uppermost undulation of the undulated bellows engages the receiving portion. The great advantage of this system is that the base carrier is first screwed to the vertebral body by means of pedicle screws for example before the elastic element, which is configured to be an undulated bellows, is introduced into the receiving portion and before the top carrier is pulled over the undulated bellows. As a result, the device is readily implanted during spine surgery.

It has thereby been found advantageous to limit the undercuts on one side by an abutment in order to prevent the undulated bellows from inadvertently sliding off at least on this side and above all in order for the surgeon to know how far he can push the undulated bellows into the base or top part. This permits to achieve accurate positioning of the undulated bellows with respect to the base and/or top part.

In an alternative, preferred embodiment, the base carrier and/or top carrier is connected to the undulated bellows through a bayonet connection. This makes it possible to readily connect the undulated bellows to the base and/or top carrier, even during surgery.

In another preferred embodiment, a vertically protruding sharp edge is formed on one side of the top carrier that is turned toward the vertebral body. The implanted device is then brought into its final position by this edge that carves into the vertebral body disposed above the top carrier, thus being secured from laterally sliding off.

In another preferred embodiment, some vertically protruding spikes are attached to the one side of the top carrier and of the base carrier that is turned toward the vertebral body, said spikes penetrating into the respective ones of the vertebral bodies and preventing the device from rotating out of position.

In still another preferred developed implementation, the top carrier is configured to have a key-shaped cross section in order for this cross section to already accommodate the curvature of the spine so that the undulated bellows may have a substantially rectilinear orientation.

In still another preferred embodiment, the top carrier is configured to be rectangular in cross section with some undulation troughs and/or undulation crests being configured in the shape of a key in order to achieve, in terms of construction, a curved undulation crest.

Further advantages of the device of the invention will become apparent from the appended drawing and from the following description of embodiments thereof. Likewise, the invention lies in each and every novel feature or combination of features mentioned above or described herein after. The embodiments discussed herein are merely exemplary in nature and are not intended to limit the scope of the invention in any manner.

### BRIEF DESCRIPTION OF THE SEVERAL VIEWS OF THE DRAWING

In the drawing:

FIG. 1 is an exploded side view of a human spine together with a first embodiment of the device of the invention;

FIG. 2 is an exploded illustration of the device according to FIG. 1;

FIG. 3 is a front view of the device of FIG. 1;

FIG. 4 is an exploded side view of a human spine together with a second embodiment of the device of the invention;

FIG. 5 is a side view of the second embodiment of a device of the invention according to FIG. 4;

FIG. 6 is a front view of the device of FIG. 4;

FIG. 7 is a side view of a third embodiment of a device of the invention;

FIG. 8 is a front view of the device of FIG. 7;

FIG. 9 is a side view of a fourth embodiment of a device of the invention;

FIG. 10 is a front view of the device of FIG. 9;

FIG. 11 is a side view of a fifth embodiment of a device of the invention;

FIG. 12 is a front view of the device of FIG. 11.

### DETAILED DESCRIPTION OF THE INVENTION

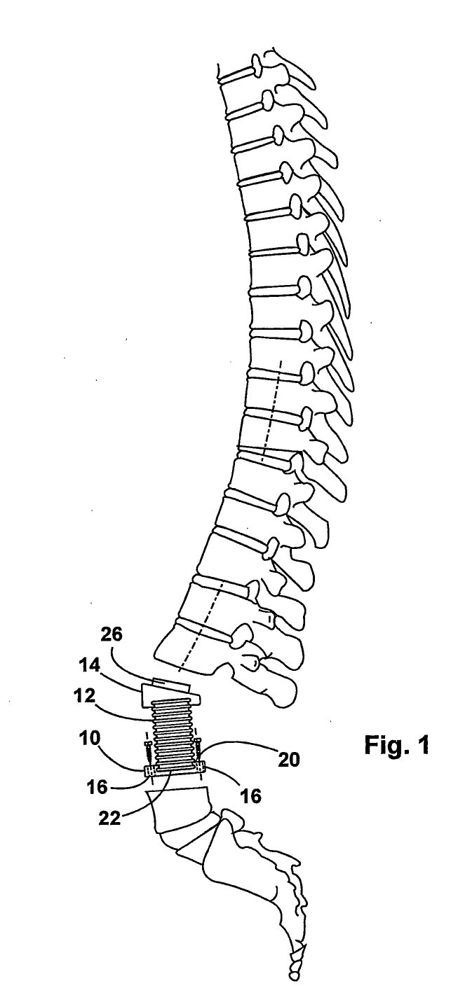

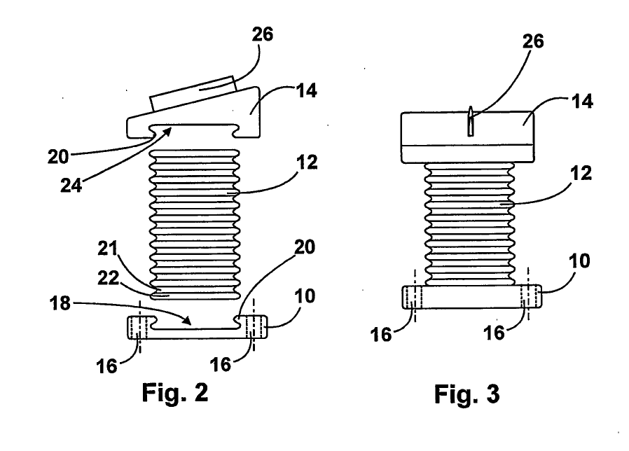

The first embodiment of a device of the invention suitable for implantation into a human or animal spine as illustrated in the FIGS. 1 through 3 includes a base carrier 10 made of a rigid material, an elastic element configured to be an undulated bellows 12 and a top carrier 14. As best shown in FIG. 2, there are provided on the base carrier openings 16 for receiving a bone screw by means of which the base carrier 10 is fastened to the vertebral body. The bone screw is manufactured from a biomaterial that is slowly resorbed over time by the body, with the top carrier 14 and the base carrier 10 having become firmly secured to the vertebral body in the meantime.

The metallic undulated bellows 12 comprises an undulated outer contour, with discrete undulation troughs 21 and crests 22 annularly surrounding the undulated bellows 12. This undulated bellows 12 resembles for example an accordion hose. The base carrier 10 further possesses, on its side turned away from the vertebral body, a receiving portion 18 for fastening the undulated bellows 12 to the base carrier 10. Said receiving portion 18 has a cross section conforming to the contour of an undulation trough 21 and of an undulation crest 22 of the undulated bellows 12 and has two crosspieces 20 that project into the receiving portion 18. The last undulation crest 22 of the undulated bellows 12 is laterally inserted into the receiving portion 18 where it is positively retained on the base carrier 10 by the crosspieces 20. On the side of the top carrier 14 that is turned toward the undulated bellows 12, there is provided a second receiving portion 24 that is configured analogous to the first receiving portion 18. Again, the undulated bellows 12 can be inserted into the receiving portion 24 by its outer undulation crest 22 in order to be positively retained therein.

The top carrier 14 is configured to have a key-shaped cross section and has a perpendicularly protruding sharp edge 26 in its center. The sharp edge 26 is oriented normal to the longitudinal direction of the receiving portion 24. The top carrier 14 and the base carrier 10 are provided with a commercially available coating in order to provide for a quick and good bond between these and the vertebral bone.

It is understood that the device of the invention is manufactured in various sizes. The size of the top carrier 14 and/or of the base carrier 10 varies so that a specimen of appropriate dimensions is available for each vertebra. The length and the elasticity of the undulated bellows 12 also vary. Only the diameter of the undulated bellows 12 and the corresponding receiving portions 18, 24 remain the same in all the devices so that every base carrier 10 may be combined with every undulated bellows 12.

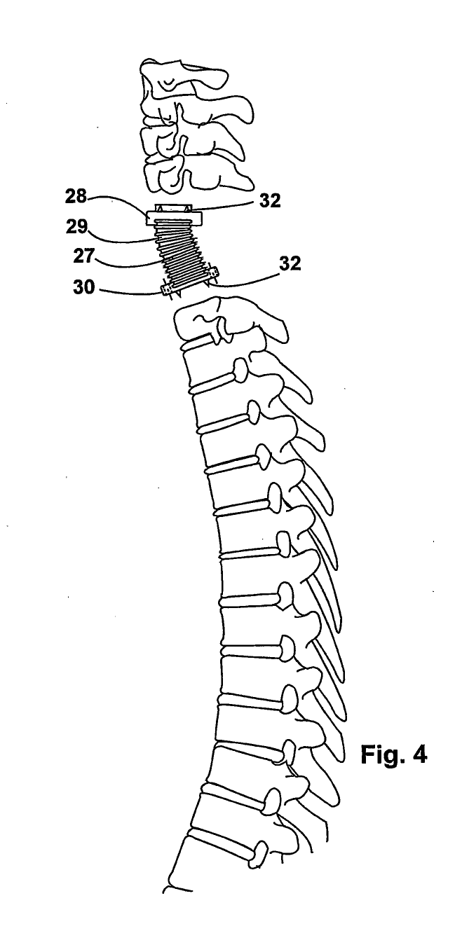

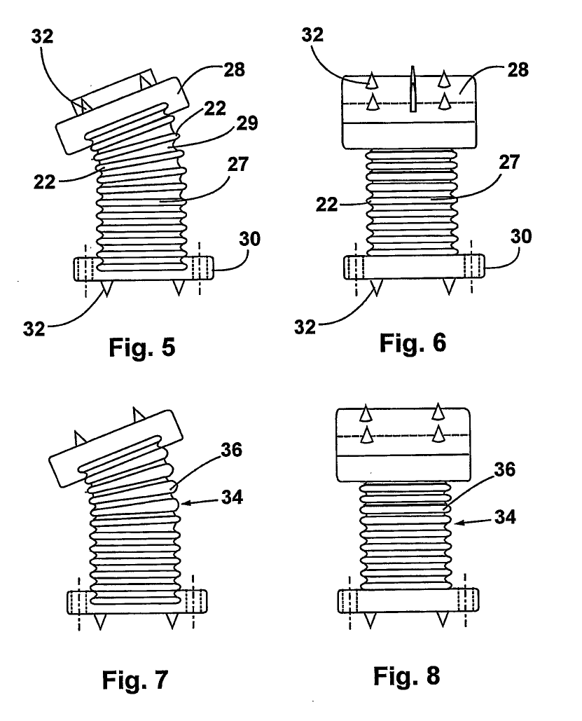

The second embodiment of the device of the invention, which is illustrated in the FIGS. 4 through 6, is substantially identical with the first embodiment illustrated in the FIGS. 1 through 3, and merely differs therefrom by the fact that in this second embodiment the undulated bellows 27 implemented as an accordion hose is configured to be slightly curved and that the top carrier 28 is not configured to be key-shaped but rectangular in cross section. The undulation troughs 29 vary in width in function of the curvature whereas the undulations 22 remain unchanged.

As a complement to the first embodiment, multiple spikes 32 are formed in this second embodiment on the sides of the top carrier 28 and of the base carrier 30 that are turned toward the vertebral bones, said spikes extending into the respective vertebral bones and preventing the device from rotating out of place. Like the bone screws, said spikes 32 are made from a resorbing biomaterial.

The third embodiment illustrated in the FIGS. 7 and 8 differs from the second embodiment illustrated in the FIGS. 4 through 6 by the fact that in this case the curvature of the undulated bellows 34 is not compensated for by the undulation troughs, but by the undulations 36. Accordingly, the undulation 36 is implemented to be narrower on the inner side of the curvature than on the outer side of the same.

The device suitable for implantation in a human or animal spine is utilized whenever an intervertebral disc between two vertebral bodies cannot be repaired and must be removed. In some cases, one or more adjacent vertebral bodies are also weak and need to be completely or partially removed as well. They will then be replaced by the device of the invention. It has been found advantageous to insert the device of the invention in the lumbar spine, in the intervertebral space between the fourth and the second lumbar vertebra, as a stiffened spine is most problematic there.

To implant the device of the invention, the operating surgeon first fastens the base carrier 10 to the lower vertebral body using for this purpose bone screws that have not been illustrated herein and then laterally inserts the undulated bellows 12 by its last undulation crest 22 into the receiving portion 18. Then, the top carrier 14 is pulled over the uppermost undulation crest 22 of the undulated bellows 12 and the undulated bellows 12 is bent to allow access. After the device has been assembled in this way, the surgeon pushes the undulated bellows 12 into the desired position with the undulated bellows 12 constituting a connection between the lower portion of the spine and the upper portion of the spine. The sharp edge 26 of the top carrier 14 is thereby brought close to the vertebral body located thereabove so that the sharp edge 26 penetrates into the same. For the rest, the top carrier 14 is fixated using most common methods.

Implanted in this way, the top carrier 14 and the base carrier 10 grow onto the respective vertebral bone, assisted therein by the coating. At the same time, the body resorbs the bone screws made from biomaterial.

It is understood that the operating surgeon will insert an undulated bellows 12 having the length and elasticity suited for each specific case.

As shown in FIG. 4, the device of the invention, if dimensioned accordingly, can also be implanted into the cervical spine in the manner described herein above.

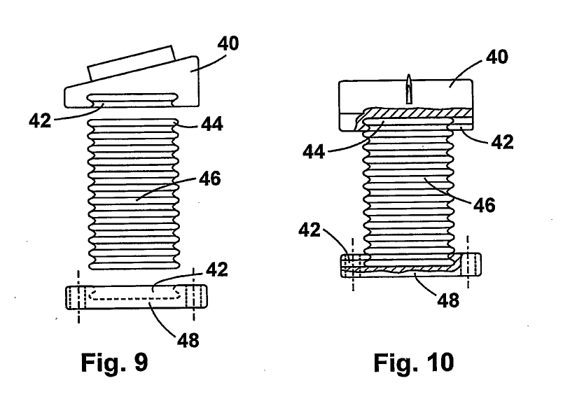

In the fourth embodiment illustrated in the FIGS. 9 and 10, an abutment 42 against which the uppermost undulation 44 comes to rest is formed in the receiving portion 24 provided on the top carrier 40. As a result, the undulated bellows 46 can be accurately positioned and mounting is facilitated since the surgeon is now capable of inserting the undulated bellows 46 into the top carrier 40 or into the base carrier 48 without having to care for other details. The abutment 42 is thereby implemented as a side wall of the top carrier 40 and possesses a receiving portion 24 the contour of which is configured to correspond to the contour of the undulation 44.

In a receiving portion 18 provided on the base part 48, there is also implemented an abutment 42 that corresponds to the abutment 42 of the top carrier 40. The abutment 42 of the base part 48 is however disposed exactly opposite the abutment of the top carrier 40 so that the two abutments 42 are virtually confronting each other.

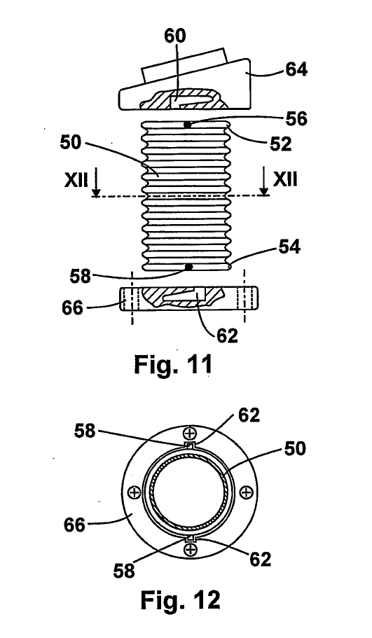

In the fifth embodiment illustrated in the FIGS. 11 and 12, two radially protruding pins 56, 58 are formed on a respective one of the uppermost and lowermost undulations 52, of the undulated bellows 50, said pins being insertable into corresponding recesses 60, 62 provided in the top carrier 64 and in the base carrier 66 respectively. The pins 56, 58 cooperate with the recesses 60, 62 to form a bayonet connection. This bayonet connection allows for fast and easy insertion of the undulated bellows 50 into the top carrier 64 or into the base carrier 66, with the pins 56, 58 being inserted into the recesses 60, 62. By pivoting the elastic element 50, the pins 56, 58 are caused to enter deep into the L-shaped recess 60, 62 where they get jammed since the recesses are configured to have a conical taper. By causing the bayonet socket to get jammed in the conical taper of the recesses 60, 62, the undulated bellows 50 is well locked in the top carrier 64 and/or in the base carrier 66 so that the connection is prevented from loosening later.

### LISTING OF NUMERALS

10 base carrier
12 undulated bellows
14 top carrier
16 opening
18 receiving portion
20 crosspiece
21 undulation trough
22 undulation crest
24 receiving portion
26 sharp edge
27 undulated bellows
28 top carrier
29 undulation trough
30 base carrier
32 spike
34, 36 undulated bellows
40 top carrier
42 abutment
44 undulation
46 undulated bellows
48 base carrier
50 undulated bellows
52, 54 undulation
56, 58 pins
60, 62 recess
64 top carrier
66 base carrier

I claim:

1. A device for implantation in a human or animal spine, said device having a base carrier (10) fastenable to a first vertebral body, a top carrier (14) fastenable to a second vertebral body and a spacer element retained between said base carrier and said top carrier, a receiving portion (18, 24) comprising undercuts and interlockingly receiving said spacer element being provided in said base carrier (10) and/or in said top carrier (14),
characterized in that the spacer element is configured to be an elastic element.

2. The device as set forth in claim 1,
characterized in that the elastic element is configured to be a metallic undulated bellows (12).

3. The device as set forth in claim 1,
characterized in that in the base carrier (66) and/or in the top carrier (64) there is provided a receiving portion (60, 62) for interlockingly receiving the parts of the undulated bellows (50).

4. The device as set forth in claim 1,
characterized in that a vertically protruding sharp edge (26) is formed on the side of the top carrier (14) that is turned toward the vertebral body.

5. The device as set forth in claim 1,
characterized in that the top carrier (14) is configured to have a key-shaped cross section.

6. The device as set forth in claim 1,
characterized in that on the side of the top carrier (28) and/or of the base carrier (30) that is turned toward the vertebral body there is provided a vertically protruding spike (32).

7. The device as set forth in claim 1,
characterized in that one single undulation trough (29) and/or one single undulation crest (36) of the undulated bellows (27, 34) is configured to have a key-shaped cross section.

8. The device as set forth in claim 1,
characterized in that an abutment (42) is provided in the receiving portion (18, 24).

9. The device as set forth in claim 8,
characterized in that the abutment (42) is disposed in the receiving portion (18) of the base carrier (48) on a side different from that on which the abutment (42) is disposed in the receiving portion of the top carrier (40).
